(19) United States
(12) Patent Application Publication
(10) Pub. No.: US 2015/0329624 A1
(43) Pub. Date: Nov. 19, 2015

(54) IGY COMPOSITION FOR USE IN CELIAC DISEASE

(71) Applicant: CHARITE - UNIVERSITATSMEDIZIN BERLIN, Berlin (DE)

(72) Inventor: Karl SKRINER, Berlin (DE)

(73) Assignee: CHARITE - UNIVERSITATSMEDIZIN BERLIN, Berlin (DE)

(21) Appl. No.: 14/727,152

(22) Filed: Jun. 1, 2015

Related U.S. Application Data
(62) Division of application No. 14/225,882, filed on Mar. 26, 2014, now abandoned.
Application No. 14/225,882 is a continuation of application No. PCT/EP2012/068914, filed on Sep. 26, 2012.

(30) Foreign Application Priority Data
Sep. 26, 2011 (DE) 102011118028.5
Oct. 6, 2011 (DE) 102011118015.3

(51) Int. Cl. C07K 16/18

(52) U.S. Cl. CPC C07K 16/18; C07K 2317/55; C07K 2317/11; C07K 2317/23

(57) ABSTRACT

The present invention relates to an IgY composition characterized in that said IgY composition contains IgY antibodies, fragments, and/or Fab fragments thereof that specifically bind to a peptide with the amino acid sequence of SEQ ID NO: 1 and to the use of this IgY composition in the therapy of celiac disease, rheumatism, and/or wheat allergy.

The present invention relates to an IgY composition characterized in that said IgY composition contains IgY antibodies, fragments, and/or Fab fragments thereof that specifically bind to a peptide comprising an amino acid sequence of SEQ ID NO: 1, and to the use of this IgY composition in the therapy of celiac disease, rheumatism, and/or wheat allergy.

Celiac disease (according to ICD-10, version WHO 2006: K90.0), also referred to as gluten-sensitive or gluten-induced enteropathy, intestinal infantilism; or nontropical or endemic sprue, gluten intolerance, or Heubner-Herter disease in adults, is a chronic disease of the small intestinal mucosa resulting from hypersensitivity to gluten, a protein found in many cereals. This intolerance remains for life, it is in part genetically determined, and its cause cannot currently be treated.

Gluten-containing or transglutaminated foods give rise to inflammation of the small intestinal mucosa with frequently extensive destruction of the intestinal epithelial cells. Nutrients are absorbed poorly and remain undigested in the bowels. Accordingly, symptoms include weight loss, diarrhea, vomiting, anorexia, fatigue, irritability, and, not least, failure to thrive during childhood. The severity of the symptoms may vary considerably, making early diagnosis more difficult. Untreated celiac disease increases the risk of non-Hodgkin's lymphoma and carcinomas of the digestive tract, such as intestinal cancer.

Meanwhile, a number of harmful peptide fragments of gluten have been identified. They all belong to the alcohol-soluble fraction (so-called prolamins) and are referred to as gliadins. In susceptible individuals, these peptide fragments result in a complex reaction of the intestinal mucosa and the immune system. Mucosal cells of the small intestine produce increasing amounts of various classes of HLA (HLA I, DR, and DQ). Certain gliadin peptides bind to the HLA-DQ2 produced in increasing amounts. This binding is increased as a result of glutamic acid formation from the amino acid glutamine which is present in the peptide in large quantities. Formation of glutamic acid is mediated by the tissue transglutaminase enzyme (tTG), in particular by tissue transglutaminase 2 (tTG2). It is formed by tTG2-modified gluten or gliadin, which is causally associated with the development of celiac disease and with the progression of the disease. As a result of this change, the corresponding section of gliadin fits better into the “pockets” of the HLA proteins. The complex of gliadin peptide and HLA-DQ2 binds to CD4+ T helper cells, causing them to produce more of various inflammation triggering messenger substances, such as interferon-γ, TNF-α, interleukin-6, and interleukin-2. Various antibodies are formed in the further process of the inflammation. In addition to antibodies against gliadin peptides themselves (gliadin antibodies, AGA), there are so-called autoantibodies against self-antigens. Tissue transglutaminase, particularly tTG2, has been identified as the primarily responsible autoantigen.

From a pathophysiological point of view, celiac disease is understood to be a mixed form of allergy and autoimmune disease based on these findings. The allergic component in the form of hypersensitivity to gliadin, an exogenous protein, represents the triggering factor while the autoimmune response to the body's own structures is responsible for the severity of the symptoms. Ultimately, the inflammatory process results in apoptosis of enterocytes, eventually leading to a more or less pronounced loss of small intestinal villi. As a result of the reduced absorption surface, the small intestinal mucosa damaged in this way is no longer capable of sufficiently transferring the supplied foods into the bloodstream.

The current treatment of celiac disease is mainly a gluten-free diet, that is, ingestion of gluten by the patient is avoided. A form of therapy that would allow the patient to ingest gluten-containing or transglutaminated foods or, if gluten-containing or transglutaminated foods were ingested inadvertently, would alleviate the consequences of such intake for the patient, is not yet known.

It is the object of this invention to reduce or avoid one or several disadvantages of prior art. More specifically, it is the object of this invention to provide new agents for the treatment of celiac disease.

This object is accomplished by providing an IgY composition that is characterized in that said IgY composition contains IgY antibodies, fragments, and/or Fab fragments thereof that specifically bind to a peptide with an amino acid sequence of SEQ ID NO: 1. The IgY composition according to the invention may contain, for example:
IgY antibodies that specifically bind to a peptide with an amino acid sequence of SEQ ID NO: 1;
fragments of such IgY antibodies that specifically bind to a peptide with an amino acid sequence of SEQ ID NO: 1; and/or
Fab fragments of such IgY antibodies that specifically bind to a peptide with an amino acid sequence of SEQ ID NO: 1.

From WO 2011/039350 A1 and DE 10 2009 045 268.0 such peptides containing or consisting of an amino acid sequence of SEQ ID NO: 1 are known as well as antibodies that bind to such peptides. WO 2011/039350 A1 and DE 10 2009 045 268.0 disclose the use of such peptides and antibodies in the diagnosis of celiac disease. Neither WO 2011/039350 Al nor DE 10 2009 045 268.0 disclose antibodies of the IgY type which specifically bind to a peptide with the amino acid sequence of SEQ ID NO: 1, let alone their suitability for use in the therapy of celiac disease.

The present invention is based on the surprising finding that immunization of egg-laying poultry with a peptide containing the amino acid sequence of SEQ ID NO: 1 results in the formation of IgY antibodies that specifically bind to tTG2-modified gliadin peptides, that is, particularly to pathological gliadin peptides in which tTG2 was used to form glutamic acid from the amino acid glutamine that is present in the gliadin peptide. As a result of the specific binding of the IgY antibodies to the modified gliadin, the respective section of the modified gliadin peptide no longer fits into the corresponding binding sites of the HLA proteins. Consequently, no complex of modified gliadin peptide and HLA-DQ2 can be formed, and the pathological signal chain is interrupted. Manifestation of the celiac disease symptoms can be prevented or at least alleviated.

It was found that the IgY composition according to the invention containing IgY antibodies, fragments, and/or Fab fragments thereof that specifically bind to a peptide comprising an amino acid sequence of SEQ ID NO: 1, is suitable for use in the therapy of celiac disease. If a celiac disease patient has ingested gluten-containing or transglutaminated foods, or if such intake can be expected, supply of the IgY composition according to the invention can prevent the patient from developing celiac disease symptoms and/or significantly alleviate the severity of the celiac disease symptoms to be expected. This means that it will for the first time be possible for patients having celiac disease to prevent or alleviate the development of celiac disease symptoms as a result of taking in gluten-containing or transglutaminated foods. A patient with celiac disease is thus given a chance to consume gluten-containing or transglutaminated foods at least in exceptional cases, such as family parties and the like, and take part in normal social life.

The present invention relates to an IgY composition. For the purposes of this invention, an IgY composition is any composition that contains IgY antibodies, fragments, and/or Fab fragments thereof. The IgY composition may comprise other ingredients.

The IgY composition according to the invention contains IgY antibodies, fragments, and/or Fab fragments thereof. IgY antibodies are a class of antibodies found in poultry, particularly in chickens, ducks, and geese, where they represent the functional equivalent to IgG antibodies in mammals. But unlike IgG, IgY antibodies do not bind to protein A or protein G, respectively, and they also do not bind to cellular Fc receptors. In addition, IgY antibodies do not activate the complement system. IgY antibodies are particularly well-suited for administration to mammals (e.g. in the form of a nutritional supplement or pharmaceutical) since IgY antibodies do not typically have an antigenic effect in mammals and will therefore not trigger a problematic immune response in the target organism, even after multiple administration.

IgY antibodies are understood to be intact, unfragmented antibodies of the IgY class having a constant Fc region and two variable Fab regions. The antigen binding sites (CDR=complementarity determining region) which give the IgY antibody its bonding specificity are located in the variable Fab regions. The structure and extraction of antibodies are known to a person skilled in the art and described in detail, for example, in Rüdiger Schade, Irene Behn,

Michael Erhard: Chicken Egg Yolk Antibodies, Production and Application. Springer-Verlag, Berlin 2001. For the purposes of this invention, the term “IgY antibody” includes both polyclonal and monoclonal IgY antibodies, which can be produced, for example, by immunizing egg-laying poultry and subsequent isolation from the egg yolk, or by using biotechnology processes such as cloning, expression, and purification. Polyclonal IgY antibodies are preferred.

Fragments of IgY antibodies are understood to be regions of intact IgY antibodies which include the antigen binding sites of the underlying antibody and show substantially the same bonding specificity as the respective intact IgY antibody. Such fragments may, for example, be produced enzymatically, e.g. by cleaving intact antibodies using suitable proteases such as trypsin. Fragments of IgY antibodies may also be produced by biotechnology processes and/or mechanical methods. The Fab fragments in which the Fc portion of the respective intact antibody has been removed represent a special form of fragments. The term “Fab fragment” includes both fragments having one Fab region and fragments having two Fab regions (so-called F(ab)₂ fragments).

The composition according to the invention preferably contains polyclonal antibodies, fragments, and/or Fab fragments thereof that specifically bind to a peptide with the amino acid sequence of SEQ ID NO: 1. The advantage of polyclonal IgY antibodies is that they can detect more than one epitope on the peptide and thus inhibit the binding of tTG2-modified gliadin to HLA proteins particularly effectively.

The IgY composition according to the invention is characterized in that it contains IgY antibodies, fragments, and/or Fab fragments thereof that specifically bind to a peptide with the amino acid sequence of SEQ ID NO: 1. The IgY composition according to the invention is characterized in that said IgY composition comprises IgY antibodies, fragments, and/or

Fab fragments thereof that can specifically bind to a peptide consisting of an amino acid sequence of SEQ ID NO: 1.

The IgY composition according to the invention preferably contains IgY antibodies, fragments, and/or Fab fragments thereof that specifically bind to a peptide with the amino acid sequence of SEQ ID NO: 1 at a K_{D}≦500nM.

It has proven to be particularly advantageous that the IgY composition according to the invention, in addition to IgY antibodies, fragments, and/or Fab fragments thereof that specifically bind to a peptide with the amino acid sequence of SEQ ID NO: 1, comprises other IgY antibodies, fragments, and/or Fab fragments thereof that specifically bind to human tTG2 of the SEQ ID NO: 5.

The IgY composition according to the invention can in principle be produced using known methods for producing specific IgY antibodies or IgY preparations or compositions, see e.g. Rüdiger Schade, Irene Behn, Michael Erhard: Chicken Egg Yolk Antibodies, Production and Application. Springer-Verlag, Berlin 2001.

The method may comprise the following steps:
Providing the antigen to be used for immunization, for example a peptide with the amino acid sequence 1 and/or 6;
Immunization of egg-laying poultry with the antigen;
Collection of the eggs of the immunized poultry;
Preparation of the IgY composition, wherein the egg yolk of the collected eggs is processed.

In the method according to the invention, a peptide containing, or consisting of, an amino acid sequence of SEQ ID NO: 1 may be used as the antigen for immunizing the egg-laying poultry. The peptide for immunization may comprise other amino acid sequences in addition to the amino acid sequence of SEQ ID NO: 1, such as amino acid sequences of SEQ ID NOs: 2, 3 and/or 4. For example, the peptide may include multiple copies of a sequence of SEQ ID NOs: 1, 2, 3, and/or 4.

An IgY composition comprising both IgY antibodies which are specific for a peptide of SEQ ID NO: 1 and IgY antibodies which can specifically bind human tTG2 may be prepared, for example, by:
Mixing IgY antibody fractions from separate immunizations, wherein a peptide containing, or consisting of, an amino acid sequence of SEQ ID NO: 1 was used in a first immunization and an antigen including at least one fragment of human tTG2 was used in a second immunization;
A mixture including a peptide of the SEQ ID NO: 1 and an antigen including at least one fragment of human tTG2 was used for immunization; or
A peptide was used for immunization, which comprises both the amino acid sequence of SEQ ID NO: 1 and at least one fragment of human tTG2, e.g. as a fusion protein.

A peptide may be used for immunization and/or for the production of the IgY composition according to the invention, which in addition to the amino acid sequence of SEQ ID NO: 1 comprises a sequence of at least 25 subsequent amino acids, preferably of at least 100 subsequent amino acids, from the sequence of human tTG2 of SEQ ID NO: 5. The regions that are parts of the peptide may in principle be selected from any portion of the tTG2 sequence of SEQ ID NO: 5. It is preferred that the peptide, in addition to one or several amino acid sequences of SEQ ID NOs: 1, 2, 3, and/or 4, additionally includes one or several partial sequences of tTG2 or one or several copies of the entire sequence of the tTG2 of SEQ ID NO: 5. In addition, the peptide for immunization and/or for the production of the IgY composition according to the invention may include other amino acids, such as a His tag, particularly a 6xHis tag. In particular, the peptide may comprise, or consist of, a sequence of SEQ ID NO: 6 [SEQ ID NOs 5+2] or of SEQ ID NO: 7 [SEQ ID NOs 5+2 +2]. One advantage of immunization with such a fusion protein is that, in a single immunization, polyclonal IgY antibody fractions can be obtained which contain both IgY antibodies that bind to the peptide of SEQ ID NO: 1 and antibodies that bind to, and block, human tTG2. In this way, a dual effect is achieved. On the one hand, pathogenic tTG2-modified gliadin is bound and prevented from binding to the patient's HLA proteins, on the other hand, the enzymatic function of tTG2 is inhibited, which prevents the formation of pathogenic tTG2-modified gliadin.

In the method according to the invention, the preferred egg-laying poultry are chickens, ducks, or geese. The use of chickens is particularly preferred. Specified pathogen-free chickens (SPF chickens) may be used to obtain a product that is as pure as possible.

The egg-laying poultry is immunized with the antigen in a generally known manner.

The eggs laid by the immunized animals are collected and used for preparing the IgY composition according to the invention.

The preparation of the IgY composition may comprise a number of various steps depending on the desired or required level of purity. First, the egg yolk containing the IgY antibody may be separated from the egg white. The egg yolk separated from the egg white can already be used for some applications as IgY composition of the invention, e.g. as an additive for foods. It is also possible to process the extracted egg yolk further to obtain purer IgY compositions. For example, the IgY antibodies can be affinity purified and/or separated from other constituents of the egg yolk using a size exclusion method. A person skilled in the art knows processes and methods for isolating specific IgY antibodies from the egg yolk and purifying the same.

The invention also relates to an IgY composition obtained using the method according to the invention.

The IgY composition according to the invention may, for example, be characterized in that said IgY composition is obtained using a method according to the invention that includes the following steps:
a) Immunization of egg-laying poultry with a peptide containing, or consisting of, an amino acid sequence of SEQ ID NO: 1;
b) Collection of the eggs of the immunized poultry; and
c) Preparation of the IgY composition, wherein the egg yolk of the collected eggs is processed.

The egg-laying poultry may be immunized with a peptide containing, or consisting of, an amino acid sequence of SEQ ID NOs: 1, 6, or 7 or with a peptide mixture comprising peptides containing an amino acid sequence of SEQ ID NO: 1 and peptides containing an amino acid sequence of SEQ ID NO: 5.

Another object of this invention are foods and food additives containing the IgY composition according to the invention.

This invention also relates to a pharmaceutical composition containing the IgY composition according to the invention and optionally one or several medically compatible adjuvants.

The pharmaceutical composition according to this invention may be of different designs depending on the desired method of administration. It is preferred that the IgY composition according to the invention or the pharmaceutical composition according to the invention, respectively, is formulated for oral administration. The formulation for oral administration may include formulations in which the IgY composition is enclosed by an enteric coating which allows the IgY composition to pass into the bowels for improved absorption. A formulation containing the IgY composition in dry or liquid form may be a capsule, such that the formulation can resist premature digestion in the acidic environment of the stomach and pass into the affected regions in the bowels.

The pharmaceutical composition according to the invention preferably includes an IgY composition according to the invention and at least one medically compatible adjuvant. The term “adjuvant” is used here for describing all other ingredients except the IgY composition according to the invention. The adjuvant is primarily selected based on the special method of administration. Adjuvants are in particular understood to be carrier substances, additives, and solvents which are physiologically compatible. Commonly used adjuvants are described in Remington's Pharmaceutical Science, 20th ed., 2000, Mack Publishing Company. The adjuvants are non-toxic at the dosages and concentrations used. Compositions for in vivo administration may be prepared using processes and methods known to a person skilled in the art.

A kit comprising a container containing an IgY composition according to the invention or a pharmaceutical composition according to the invention and instructions for use is also a part of this invention.

The IgY composition or pharmaceutical composition according to the invention may be used in the therapy of celiac disease, rheumatism, and/or wheat allergy. In all these diseases, tTG2-modified gliadin plays a major part.

For the purposes of this invention, the term “therapy” denotes the reversal, alleviation, or inhibition of the progression of a disease, disorder, or condition or of one or several symptoms of such a disease, disorder, or condition. The term “therapy” may also refer to reducing the probability, severity, or frequency of occurrence of a disease, disorder, or condition in a mammal as compared to an untreated control population or to the same mammal prior to treatment. The term “therapy” may also refer to the prevention of a disease, disorder, or condition and may include the delay or prevention of the onset of a disease, disorder, or condition or the delay or prevention of symptoms associated with a disease, disorder, or condition. The term “therapy” may also refer to the reduction of the severity of a disease, disorder, or condition or of symptoms associated with such disease, disorder, or condition.

The use of the IgY composition or pharmaceutical composition according to the invention for the therapy of celiac disease may be characterized in that the composition according to the invention is administered by the patient (so-called self-administration). This ensures that the patient will only be exposed to a medication when he or she really needs it. The patient typically knows in advance if he or she will be involved in a situation in which the ingestion of gluten-containing or transglutaminated foods can be expected. In most cases, the patient is best positioned to notice if he or she has taken in gluten-containing or transglutaminated foods. In all these cases, a patient can independently react by administering a dose of the IgY composition or pharmaceutical composition according to the invention. Preferred is any self-administration triggered by the patient's expected or actual consumption of gluten-containing or transglutaminated foods. Self-administration should preferably take place in a time window of no more than 5 hrs before and no more than 5 hrs after the expected or actual consumption of a gluten-containing or transglutaminated food to achieve a particularly useful effect.

This invention relates to a method for treating celiac disease, rheumatism, and/or wheat allergy, wherein an effective dose of the IgY composition or pharmaceutical composition according to the invention is administered to a patient.

According to this invention, the IgY composition or pharmaceutical composition according to the invention is preferably administered at an effective dose. An “effective dose” is a dose of the composition according to the invention that causes a measurable therapeutic effect with respect to the disease in question when administered to a patient. In the present invention, an effective dose is that does of the composition according to the invention that, when administered to a patient, produces a therapeutic effect with respect to the treatment of celiac disease, rheumatism, and/or wheat allergy. The composition is preferably administered at a dose not higher than 5 mg/kg body weight per treatment or administration. In particular, the composition according to the invention may be administered at a dose from 0.1 μg/kg to 5000 μg/kg body weight per treatment or administration, preferably 1 μg/kg to 2000 μg/kg body weight per treatment or administration. It is recommended to use the composition according to the invention at a maximum cumulative daily dose of not more than 10 mg/kg body weight to prevent the occurrence of acute side effects.

### BRIEF DESCRIPTION OF THE DRAWINGS

FIG. 1 shows the inhibition of the celiac disease-specific IgA and IgG serum antibodies of 15 celiac disease patients by anti-tTG-CD3-IgY and anti-CD3-IgY chicken antibodies.

FIG. 2 shows the specific binding of anti-tTG-CD3-IgY and CD3-IgY to tTg2-modified wheat gliadin.

FIG. 3 shows the specific inhibition of the enzymatic activities of tTg2 by anti-tTG-CD3-IgY.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding application No. DE 102011 118 028.5, filed Sep. 26, 2011 and DE 102011 118 015.3, filed Oct. 6, 2011 are incorporated by reference herein.

### EXAMPLES

### Preparation and Purification of CD3, tTG, and CD3tTG

CD3 of SEQ ID NO: 4 was prepared by synthesis and biotinylated at the C-terminal lysine residue.

Human tTG2 of SEQ ID NO: 5, hereinafter referred to as tTG, was cloned in a vector with a removable 6xHis tag for recombinant expression and expressed according to a standard protocol, isolated using Ni—NTA column purification, and the 6xHis tag was removed.

CD3tTG of SEQ ID NO: 6, hereinafter referred to as CDtTG, was likewise cloned in a vector with C-terminal 6xHis tag for recombinant expression and expressed according to a standard protocol, isolated using Ni—NTA column purification, and the His tag was removed.

### Serum inhibition test

Inhibition of the celiac disease-specific IgA and IgG serum antibodies of 15 celiac disease patients by anti-tTG-CD3-IgY and anti-CD3-IgY chicken antibodies was examined. A CD3 peptide ELISA was developed for the inhibition of the CD3-specific antibodies in which biotinylated CD3 was initially coupled to a neutrAvidin-coated microtiter plate, followed by the protocol below:

### NeutrAvidin-CD3 peptide ELISA

The wells of the microtiter plate were initially blocked with PBS/5% MP buffer overnight. Then the biotinylated peptides were applied using 500 pmol/well in PBS buffer each time. After incubation for 2 hours, the CDR3/neutrAvidin-coated plates were washed 4 times with PBS/0.1% Tween and incubated for 1 hour with patient serum at a dilution of 1:800 in PBS/2% MP. Thereafter, washing was repeated 4 times, followed by application of peroxidase-conjugated second Ab at a dilution of 1:5000. Incubation also took 1 hour.

Finally, washing was repeated 4 times, followed by application of the substrate. The reaction with development of blue color was quenched after 5 minutes using 0.5 M sulfuric acid. The resulting yellow color was measured photometrically using the ELISA reader at a measurement wavelength of 450 nm versus a reference wavelength of 620 nm and visualized using the Magellan software. Each solution was applied using 100 μL per well. Blocking and each of the washing steps were performed using 300 μL per well each time. The microtiter plates with peptide, patient serum, and the 2nd Ab were incubated with agitation at RT.

For the blocking tests, the plates were incubated with 5 μg IgY (corresponds to approx. 0.5-1μg of specific CD3 antibody) in 100 μL PBS for 30 minutes after the CD3 coating and then washed 4 times with PBS/0.1% Tween.

### tTG-ELISA Protocol

For detecting tTG2-IgY inhibition antibodies, a specific ELISA was developed, wherein recombinant tTG2 was initially coupled to a MaxiSorb microtiter plate (Nunc), and the following protocol was subsequently used: A) Coupling buffer: 100 mM Tris, 10 mM NaCl pH 7.8 B) Wash buffer: 50 mM Tris-HCl, 150 mM NaCl, 10 mM EDTA, 0.1% Tween 20 pH 7.4 C) Saturation buffer 50 mM Tris-HCl, 150 mM NaCl, 0.5% BSA, 3% sucrose pH 7.4 D) Serum dilution buffer: 50 mM Tris-HCl, 150 mM NaCl, 0.5% Tween 20 pH 7.4

### Procedure

Coating on MaxiSorp plates from Nunc: Coating quantity: tTG2 was used at a concentration of 0.5 pg/well each time. Coating volume: 100 μL/well. All tTGs were suitably diluted in coupling buffer A. For coating, the plates were incubated at 4° C. overnight. Blank and 2nd Ab controls were carried along in ELISA implementation. The OD values of the blank and 2nd Ab controls were subtracted in ELISA evaluation. The plates were washed after coating with 3×300 μL/well wash buffer B. Each wash step is equivalent to 600 rpm on the ELISA shaker for 3 minutes. Thereafter the plates were blocked with 300 μL/well saturation buffer for 2 hours at RT. For the blocking tests, the plates were incubated with 5 μg IgY (corresponds to approx. 0.5-1 μg of specific CD3tTG antibody) diluted in 100 μL buffer D for 30 minutes after the tTG coating and then washed with buffer B.

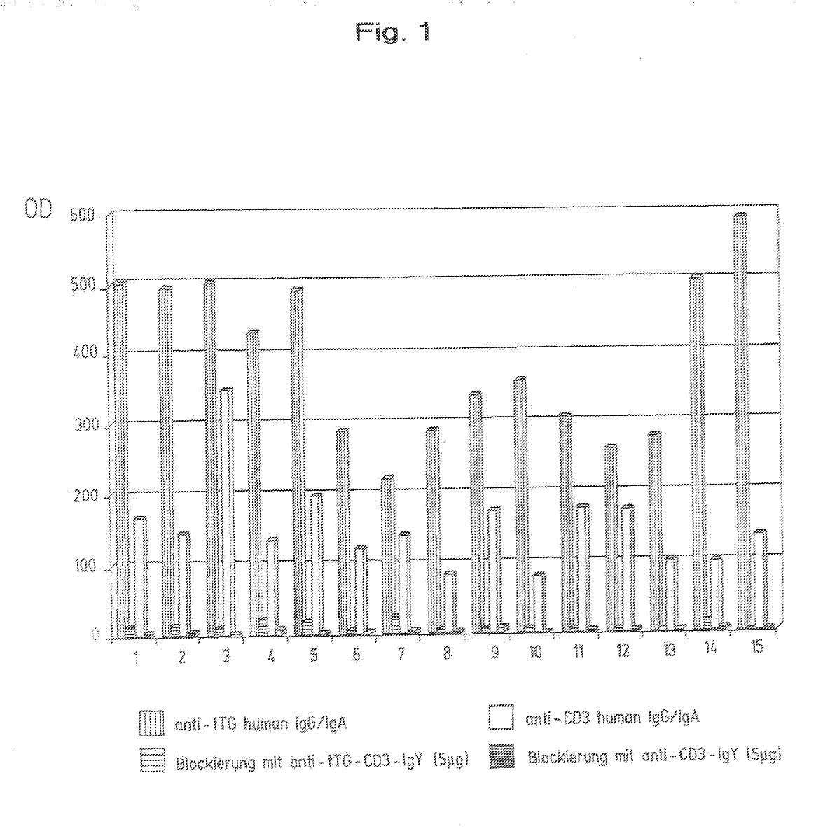

The sera were diluted 1:800 in serum dilution buffer D and 100 pUwell directly placed on the plates after blocking. Incubation at RT for 1 hour with shaking; washing with 5×300 μL/well wash buffer B. 2nd Ab:—<hlgA HRP by Dako was diluted 1:1500 in wash buffer B, and 100 pUwell were used;—<hlgG>HRP by Dako at a dilution of 1:5000, likewise in wash buffer B, and 100 μL/well were used. Incubation for 1 hour at RT with shaking. Washing with 4×300 pUwell wash buffer B. Allowing reaction with 100 μL/well TMB substrate (SeramunBlue fast) for 5 minutes. Thereafter, quenching with 100 μL/well quenching solution (0.5 M H2SO4) and evaluation in the ELISA reader at 450 nm.
3rd cohort of celiac disease patient sera In the course of the present work, human patient sera from 15 patients with positive celiac diagnosis, varying age, gender, and pathological characteristics were employed. The anti-tTG2 and -CD3 signals of all sera used were inhibited under cutoff, see FIG. 1.

### IgY Purification

The optimized chloroform polyethylene glycol protocol according to Gamenisch et al. was used for improved and faster purification of the IgY antibodies of the recombinant fusion proteins from chicken eggs. In a first step, the egg yolk is separated from the egg white. The liquid inner egg yolk is then separated off on sterile filter paper by perforating the yolk sac with a pipette and transferred into a 50 mL falcon tube. The 50 mL falcon tube with the transferred egg yolk is then filled up to 25 mL with PBS buffer and vortexed. The egg yolk-PBS mixture is then mixed with 20 mL chloroform, vortexed and shaken until a homogeneous mixture is obtained. After centrifuging at 1200×g for 10 minutes, the supernatant is transferred into a centrifuge tube and set to a final concentration of 12% (w/v) using polyethylene glycol 6000 (Fluka). After centrifuging at 15700 xg for 10 minutes, the pellet is resuspended with PBS, a portion is checked for reactivity with the respective protein or peptide in a Western blot test or ELISA, the rest is stored in a cryotube vial at −80° C.

### Preparation of Polyclonal IgY Antibodies Against CD3tTG and CD3

Chickens were selected for preparing polyclonal antibodies because large quantities of IgY antibodies can be isolated from the egg yolk Y, and only smaller antigen quantities are needed for immunization. 100 to 150 μg of antigen (CD3 peptide coupled to KLH and recombinant tTG-CD3), which were present in 250 uL elution buffer, were used per immunization step. This portion was mixed in equal parts with Freund's adjuvant. The chickens were stimulated for producing antibodies by intramuscular injection with the recombinant fusion proteins purified 2 times over HisPurTM cobalt resin and 1 time over Q Sepharose® Fast Flow. Booster vaccinations were given after 14 and 38 days. The chicken eggs were collected after a total of 64 days.

The purchased wheat gliadin (Sigma) was also coupled to the wells (2 ug). The gliadin was dissolved in 8 M urea (60 μg/mL), and the respective dilutions were prepared. After coupling, the proteins were enzymatically modified by transglutaminase. The transglutaminase catalyzes the deamination of glutamine. This results in the transformation of glutamine into glutamate. The ELISA plate was washed with PBS buffer for the modification. After adding 100 uL modification buffer, 20 μL tTG2 (250 μg/mL) were added by pipetting, deposited in quantities of 120 μL/well at 37° C. and incubated for 2 hours. A 2×1 minute washing step with PBS followed.

### Modification buffer

10 mM DTT
10 mM CaCl₂
in TBS

Subsequent blocking for 1 hour was performed with 150 μL each of 5% milk powder/PBS. A 2×1 minute washing step with PBS followed. 120 μL anti-CD3 and anti-CD3-tTG IgY antibodies were each diluted in 5% milk powder at 1:500 and 1:1000, pipetted into the wells and incubated for 1 hour at RT. After a washing step of 4×1 minutes with PBS, 120 μL of HRP-coupled secondary antibody were added (anti-chicken antibody 1:2000 for anti-CD3 and CD3tTG) in 5% milk powder. Incubation was performed for 30 minutes. Then the product was washed 5×1 minutes with PBS. The immune reaction was visualized by respectively adding 100 μL TMB for 5 minutes and subsequently stopping the reaction using 100 μL 0.5 M H₂SO₄. The result was measured at 450 nm against a reference wavelength of 620 nm at the ELISA plate photometer. In all ELISA tests performed, the incubation and washing steps were performed on a shaker (Titramax 1400, Heidolph) at 600 rpm. Every analysis was performed in duplicate and subsequent controls were included.

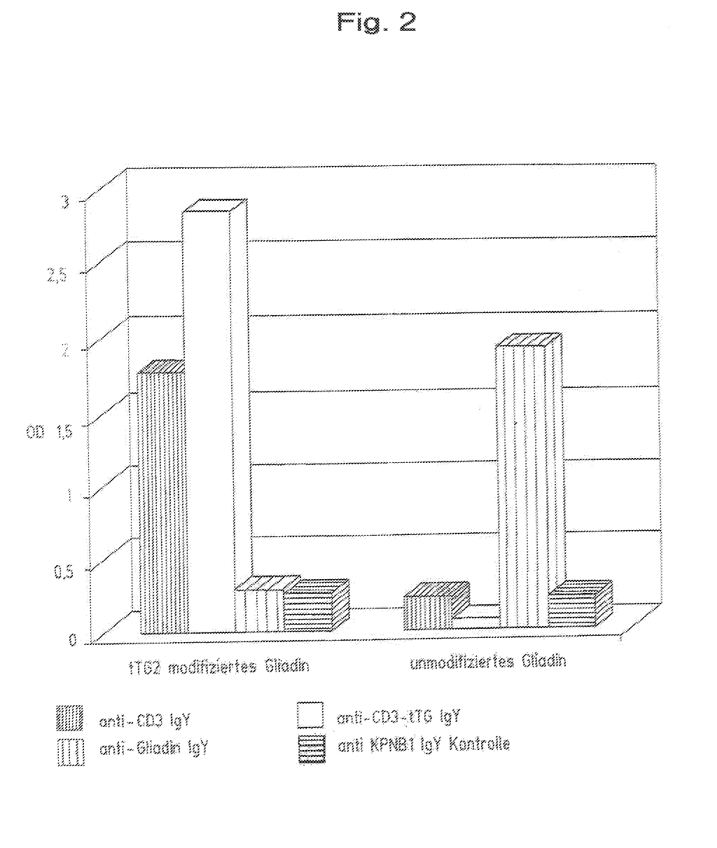

The results are shown in FIG. 2. Anti-CD3 IgY and anti-CD3-tTG IgY antibodies specifically detect the tTG-modified form of wheat gliadin while the anti-CD3 IgY and anti-CD3-tTG IgY antibodies do not show a specific reactivity with non-pathogenic, unmodified gliadin. An IgY antibody directed against unmodified gliadin shows a specific binding to an unmodified gliadin while tTG2-modified gliadin is not detected by this antibody.

### tTG2 Enzyme Activity Test

The enzymatic activity of tTG in the presence and absence of anti-CD3-tTG IgY antibodies was quantified by measuring the incorporation of casein. Casein (17 mM) was incubated with 30 mM monodansyl cadaverine at a total volume of 100 mL 0.1 M Tris-HCl, 0.15 M NaCl, 5 mM CaCl2, pH 7.5. The incorporation of monodansyl cadaverine (N-(5-aminopentyl)-5-dimethylamino-1-naphthalene sulfonamide by Sigma into bovine a-Casein results in increased intensity of the fluorescence of the dansyl group. For inhibition studies, the anti-CD3-tTG IgY antibodies were included at various concentrations in the range from 1 and 2,5 mg. The reaction was started by adding 0.5 mg human recombinant tTG at 37° C. Excitation took place at 360 nm and the increase in fluorescence was measured at 550 nm using a fluorescence spectrophotometer. tTG activity was calculated as a percentage of the remaining activity in correlation to the experiments that involved the addition of control antibodies.

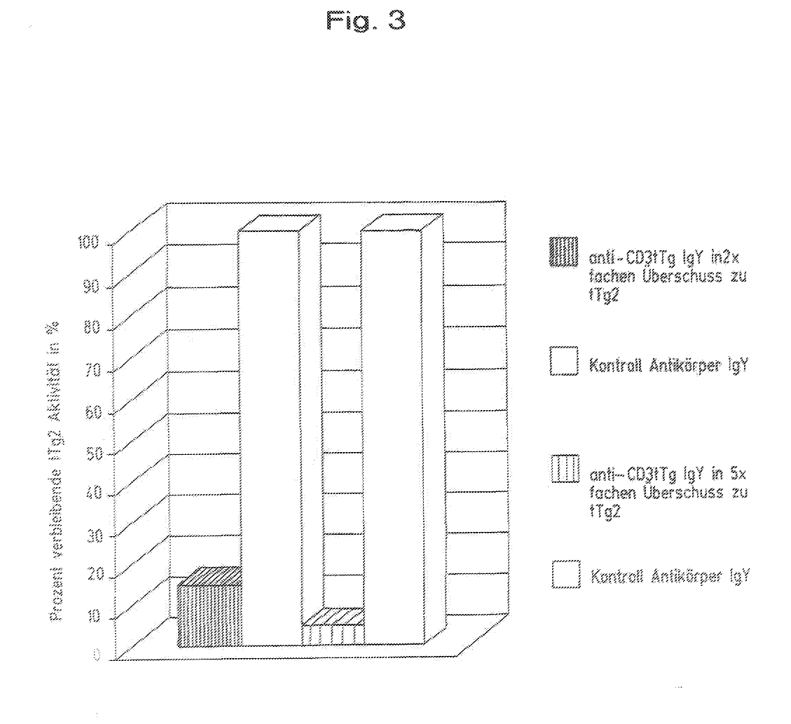

As shown in FIG. 3, the anti-CD3-tTG IgY antibody is capable of effectively blocking tTG2 while an irrelevant IgY control antibody has no influence on the tTG2 activity.

Anti-CD3-tTG IgY showed >95% inhibition of the tTG2 activity. This inhibitory effect enables anti-CD3-tTG IgY antibodies to block the pathological activation and reproduction of T cells.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1-14. (canceled)

15. A method for therapy of celiac disease, rheumatism and/or wheat allergy, comprising administering to a patient in need thereof an effective amount of an IgY composition, wherein said IgY composition comprises an IgY antibody, a fragment thereof, and/or an Fab fragment thereof, that specifically bind to a peptide having an amino acid sequence of SEQ ID NO: 1, 2, 3 and/or 4.

16. The method of claim 15, wherein said IgY composition comprises polyclonal IgY antibodies, fragments, and/or Fab fragments thereof that specifically bind to a peptide having the amino acid sequence of SEQ ID NO: 1, 2, 3 and/or 4.

17. The method of claim 15, wherein said IgY composition comprises IgY antibodies, fragments, and/or Fab fragments thereof that specifically bind to a peptide having the amino acid sequence of SEQ ID NO: 1, 2, 3 and/or 4 at a K_{D}≦500 nM.

18. The method of claim 15, wherein said IgY composition is obtained using a method comprising:
a) immunizing egg-laying poultry with a peptide comprising an amino acid sequence of SEQ ID NO: 1;
b) collecting the eggs of the immunized poultry; and
c) preparing the IgY composition, wherein the egg yolk of the collected eggs is processed.

19. The method of claim 18, wherein the immunization of the egg-laying poultry is performed with a peptide comprising an amino acid sequence of SEQ ID NOs: 1, 2, 3, 4, 6, or 7 or with a mixture of peptides comprising peptides comprising an amino acid sequence of SEQ ID NO: 1 and peptides comprising an amino acid sequence of SEQ ID NO: 5.

20. The method according to claim 18, wherein the step of processing the egg yolk includes isolating IgY antibodies from the egg yolk.

21. The method of claim 18, wherein the poultry is chicken.

22. The method of claim 15, wherein the IgY composition is self-administered by the patient.

23. The method of claim 15, wherein the IgY composition is self-administered by the patient, and each self-administration is triggered by the expected or actual consumption of a gluten-containing or transglutaminated food.

24. The method of claim 15, wherein the IgY composition is self-administered by the patient, and each self-administration takes place in a time window of no more than five hours before and no more than five hours after the expected or actual consumption of a gluten-containing or transglutaminated food.

25. The method of claim 18, wherein the immunization of the egg-laying poultry is performed with a peptide consisting of an amino acid sequence of SEQ ID NOs: 1, 2, 3, 4, 6, or 7 or with a mixture of peptides consisting of peptides consisting of an amino acid sequence of SEQ ID NO: 1 and peptides consisting of an amino acid sequence of SEQ ID NO: 5.

26. The method of claim 21, wherein the poultry is SPF chicken.


SEQUENCE LISTING (7 sequences)

SEQ ID NO: 1 - protein, 24 aa, Artificial Sequence
  Description of Artificial Sequence Synthetic peptide
PFPQPEQPFQPEQPFQEQPFPQPE

SEQ ID NO: 2 - protein, 30 aa, Artificial Sequence
  Description of Artificial Sequence Synthetic polypeptide
QEQPFPQPEQPFQPEQPFQEQPFPQPEQPF

SEQ ID NO: 3 - protein, 31 aa, Artificial Sequence
  Description of Artificial Sequence Synthetic polypeptide
QEQPFPQPEQPFQPEQPFQEQPFPQPEQPFK

SEQ ID NO: 4 - protein, 31 aa, Artificial Sequence; identical to SEQ ID NO: 3
  Description of Artificial Sequence Synthetic polypeptide

SEQ ID NO: 5 - protein, 687 aa, Homo sapiens
MAEELVLERCDLELETNGRDHHTADLCREKLVVRRGQPFWLTLHFEGRNYEASVDSLTFSVVTGPAPSQEAGTKARFPLRDAVEEGDWTATVVDQQDCTLSLQLTTPANAPIGLYRLSLEASTGYQGSSFVLGHFILLFNAWCPADAVYLDSEEERQEYVLTQQGFIYQGSAKFIKNIPWNFGQFEDGILDICLILLDVNPKFLKNAGRDCSRRSSPVYVGRVVSGMVNCNDDQGVLLGRWDNNYGDGVSPMSWIGSVDILRRWKNHGCQRVKYGQCWVFAAVACTVLRCLGIPTRVVTNYNSAHDQNSNLLIEYFRNEFGEIQGDKSEMIWNFHCWVESWMTRPDLQPGYEGWQALDPTPQEKSEGTYCCGPVPVRAIKEGDLSTKYDAPFVFAEVNADVVDWIQQDDGSVHKSINRSLIVGLKISTKSVGRDEREDITHTYKYPEGSSEEREAFTRANHLNKLAEKEETGMAMRIRVGQSMNMGSDFDVFAHITNNTAEEYVCRLLLCARTVSYNGILGPECGTKYLLNLNLEPFSEKSVPLCILYEKYRDCLTESNLIKVRALLVEPVINSYLLAERDLYLENPEIKIRILGEPKQKRKLVAEVSLQNPLPVALEGCTFTVEGAGLTEEQKTVEIPDPVEAGEEVKVRMDLLPLHMGLHKLVVNFESDKLKAVKGFRNVIIGPA

SEQ ID NO: 6 - protein, 717 aa, Artificial Sequence
  Description of Artificial Sequence Synthetic polypeptide
MAEELVLERCDLELETNGRDHHTADLCREKLVVRRGQPFWLTLHFEGRNYEASVDSLTFSVVTGPAPSQEAGTKARFPLRDAVEEGDWTATVVDQQDCTLSLQLTTPANAPIGLYRLSLEASTGYQGSSFVLGHFILLFNAWCPADAVYLDSEEERQEYVLTQQGFIYQGSAKFIKNIPWNFGQFEDGILDICLILLDVNPKFLKNAGRDCSRRSSPVYVGRVVSGMVNCNDDQGVLLGRWDNNYGDGVSPMSWIGSVDILRRWKNHGCQRVKYGQCWVFAAVACTVLRCLGIPTRVVTNYNSAHDQNSNLLIEYFRNEFGEIQGDKSEMIWNFHCWVESWMTRPDLQPGYEGWQALDPTPQEKSEGTYCCGPVPVRAIKEGDLSTKYDAPFVFAEVNADVVDWIQQDDGSVHKSINRSLIVGLKISTKSVGRDEREDITHTYKYPEGSSEEREAFTRANHLNKLAEKEETGMAMRIRVGQSMNMGSDFDVFAHITNNTAEEYVCRLLLCARTVSYNGILGPECGTKYLLNLNLEPFSEKSVPLCILYEKYRDCLTESNLIKVRALLVEPVINSYLLAERDLYLENPEIKIRILGEPKQKRKLVAEVSLQNPLPVALEGCTFTVEGAGLTEEQKTVEIPDPVEAGEEVKVRMDLLPLHMGLHKLVVNFESDKLKAVKGFRNVIIGPAQEQPFPQPEQPFQPEQPFQEQPFPQPEQPF

SEQ ID NO: 7 - protein, 747 aa, Artificial Sequence
  Description of Artificial Sequence Synthetic polypeptide
MAEELVLERCDLELETNGRDHHTADLCREKLVVRRGQPFWLTLHFEGRNYEASVDSLTFSVVTGPAPSQEAGTKARFPLRDAVEEGDWTATVVDQQDCTLSLQLTTPANAPIGLYRLSLEASTGYQGSSFVLGHFILLFNAWCPADAVYLDSEEERQEYVLTQQGFIYQGSAKFIKNIPWNFGQFEDGILDICLILLDVNPKFLKNAGRDCSRRSSPVYVGRVVSGMVNCNDDQGVLLGRWDNNYGDGVSPMSWIGSVDILRRWKNHGCQRVKYGQCWVFAAVACTVLRCLGIPTRVVTNYNSAHDQNSNLLIEYFRNEFGEIQGDKSEMIWNFHCWVESWMTRPDLQPGYEGWQALDPTPQEKSEGTYCCGPVPVRAIKEGDLSTKYDAPFVFAEVNADVVDWIQQDDGSVHKSINRSLIVGLKISTKSVGRDEREDITHTYKYPEGSSEEREAFTRANHLNKLAEKEETGMAMRIRVGQSMNMGSDFDVFAHITNNTAEEYVCRLLLCARTVSYNGILGPECGTKYLLNLNLEPFSEKSVPLCILYEKYRDCLTESNLIKVRALLVEPVINSYLLAERDLYLENPEIKIRILGEPKQKRKLVAEVSLQNPLPVALEGCTFTVEGAGLTEEQKTVEIPDPVEAGEEVKVRMDLLPLHMGLHKLVVNFESDKLKAVKGFRNVIIGPAQEQPFPQPEQPFQPEQPFQEQPFPQPEQPFQEQPFPQPEQPFQPEQPFQEQPFPQPEQPF